(19) United States
(12) Patent Application Publication
(10) Pub. No.: US 2025/0295314 A1
(43) Pub. Date: Sep. 25, 2025

(54) SYSTEMS AND METHODS FOR DETERMINING SYSTEMIC VASCULAR RESISTANCE USING PHOTOPLETHYSMOGRAPHY

(71) Applicant: BaroPace, Inc., Issaquah, WA (US)

(72) Inventor: Michael BURNAM, Issaquah, WA (US)

(73) Assignee: BaroPace, Inc., Issaquah, WA (US)

(21) Appl. No.: 18/992,847

(22) PCT Filed: Jul. 13, 2023
(86) PCT No.: PCT/US2023/070135

§ 371 Date: Jan. 9, 2025

Related U.S. Application Data
(60) Provisional application No. 63/368,316, filed on Jul. 13, 2022.

(51) Int. Cl. A61B 5/02; A61B 5/00; A61B 5/0205; A61B 5/021; A61B 5/024; A61B 5/029; A61B 5/346; G16H 50/20; G16H 50/70

(52) U.S. Cl. CPC A61B 5/02028; A61B 5/0205; A61B 5/02125; A61B 5/024; A61B 5/029; A61B 5/346; A61B 5/686; A61B 5/7207; G16H 50/20; G16H 50/70; A61B 2560/0223

(57) ABSTRACT

Methods, systems, and computer-readable medium for determining a systemic vascular resistance (SVR), by receiving a systolic blood pressure (SBP) and a diastolic blood pressure (DPB) from a calibrated photoplethysmography (PPG) device, receiving a pulse transit time (PTT) from the PPG device, determining a stroke volume based on the PTT, determining a mean arterial pressure (MAP) based on the SBP and the DBP, receiving a heart rate, determining a cardiac output based on the heart rate and the stroke volume, determining a first value based on a right atrial pressure (RAP) or central venous pressure (CVP) and the map, determining a second value based on the first value and the cardiac output, and determining a SVR based on the second value and a factor.

### CROSS-REFERENCE TO RELATED APPLICATIONS

This patent application claims the benefit of U.S. Provisional Application No. 63/368,316, filed on Jul. 13, 2022, the entirety of which is incorporated by reference herein.

### TECHNICAL FIELD

Various embodiments of the present disclosure relate generally to systemic vascular resistance (SVR) determination, and, more specifically, to using a photoplethysmography (PPG) device to determine SVR.

### INTRODUCTION

The total resistance to blood flow through peripheral vascular beds has an influence on cardiac output. A rise in total peripheral resistance can raise arterial blood pressure which may reduce cardiac output. A fall in total peripheral resistance may increase cardiac output. A number of human disease states are associated with abnormal systemic vascular resistance (SVR), such as heart failure, hypertension, autoimmune disorders associated with inflammation of blood vessels, systemic infection, shock, etc.

The introduction description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

According to certain aspects of the disclosure, methods and systems are disclosed for determining systemic vascular resistance (SVR) using a photoplethysmography (PPG) device.

In one aspect, an exemplary embodiment of a method for determining a systemic vascular resistance (SVR), the method comprising: receiving a systolic blood pressure (SBP) and a diastolic blood pressure (DBP) from a calibrated photoplethysmography (PPG) device; receiving a pulse transit time (PTT) from the PPG device; determining a stroke volume based on the PTT; determining a mean arterial pressure (MAP) based on the SBP and the DBP; receiving a heart rate; determining a cardiac output based on the heart rate and the stroke volume; determining a first value based on a right atrial pressure (RAP) or central venous pressure (CVP) and the MAP; determining a second value based on the first value and the cardiac output; and determining a systemic vascular resistance (SVR) based on the second value and a factor.

In another aspect, an exemplary embodiment of a system for determining a systemic vascular resistance (SVR) using photoplethysmography (PPG), the system comprising: at least one memory storing instructions; and at least one processor executing the instructions to perform a process, the at least one processor configured to: receiving a systolic blood pressure (SBP) and a diastolic blood pressure (DBP) from a calibrated photoplethysmography (PPG) device; receiving a pulse transit time (PTT) from the PPG device; determining a stroke volume based on the PTT; determining a mean arterial pressure (MAP) based on the SBP and the DBP; receiving a heart rate; determining a cardiac output based on the heart rate and the stroke volume; determining a first value based on a right atrial pressure (RAP) or central venous pressure (CVP) and the MAP; determining a second value based on the first value and the cardiac output; and determining a systemic vascular resistance (SVR) based on the second value and a factor.

In a further aspect, an exemplary embodiment of a method for determining a systemic vascular resistance (SVR), the method comprising: determining a mean arterial pressure (MAP) based on a systolic blood pressure (SBP) and a diastolic blood pressure (DBP) received from a calibrated photoplethysmography (PPG) device; receiving a heart rate from a pacemaker and a pulse transit time (PTT) from the PPG device; determining a stroke volume based on the PTT; determining a cardiac output (CO) based on the heart rate and the stroke volume; determining a first value based on a right atrial pressure (RAP) or central venous pressure (CVP) and the MAP; determining a second value based on the first value and the CO; and determining the SVR based on the second value and a factor.

### BRIEF DESCRIPTION OF THE FIGURES

The accompanying drawings, which are incorporated in and constitute a part of this specification, illustrate various examples and, together with the description, serve to explain the principles of the disclosed examples and embodiments.

Aspects of the disclosure may be implemented in connection with embodiments illustrated in the attached drawings. These drawings show different aspects of the present disclosure and, where appropriate, reference numerals illustrating like structures, components, materials, and/or elements in different figures are labeled similarly. It is understood that various combinations of the structures, components, and/or elements, other than those specifically shown, are contemplated and are within the scope of the present disclosure. Moreover, there are many embodiments described and illustrated herein.

FIG. 1 depicts an exemplary environment for determining a systemic vascular resistance (SVR) using photoplethysmography (PPG), according to one or more embodiments.

FIG. 2 depicts a flowchart of an exemplary method for determining an SVR using PPG, according to one or more embodiments.

FIG. 3 depicts an example of training a machine learning model for determining the SVR, according to one or more embodiments.

FIG. 4 depicts an example of a computing device for determining the SVR, according to one or more embodiments.

Notably, for simplicity and clarity of illustration, certain aspects of the figures depict the general structure and/or manner of construction of the various embodiments. Descriptions and details of well-known features and techniques may be omitted to avoid unnecessarily obscuring other features. Elements in the figures are not necessarily drawn to scale; the dimensions of some features may be exaggerated relative to other elements to improve understanding of the example embodiments. For example, one of ordinary skill in the art appreciates that the side views are not drawn to scale and should not be viewed as representing proportional relationships between different components. The side views are provided to help illustrate the various components of the depicted assembly, and to show their relative positioning to one another.

### DETAILED DESCRIPTION

Reference will now be made in detail to examples of the present disclosure, which are illustrated in the accompanying drawings. The present disclosure is neither limited to any single aspect or embodiment thereof, nor is it limited to any combinations and/or permutations of such aspects and/or embodiments. Moreover, each of the aspects of the present disclosure, and/or embodiments thereof, may be employed alone or in combination with one or more of the other aspects of the present disclosure and/or embodiments thereof. For the sake of brevity, certain permutations and combinations are not discussed and/or illustrated separately herein.

The term “exemplary” is used in the sense of “example” rather than “ideal.” Notably, an embodiment or implementation described herein as “exemplary” is not to be construed as preferred or advantageous, for example, over other embodiments or implementations; rather, it is intended to reflect or indicate the embodiment(s) is/are “example” embodiment(s). Wherever possible, the same reference numbers will be used throughout the drawings to refer to the same or like parts. In the discussion that follows, relative terms such as “about,” “substantially,” “approximately,” etc. are used to indicate a possible variation of ±10% in a stated numeric value.

As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” In addition, the terms “first,” “second,” and the like, herein do not denote any order, quantity, or importance, but rather are used to distinguish an element or a structure from another. Moreover, the terms “a” and “an” herein do not denote a limitation of quantity, but rather denote the presence of one or more of the referenced items. In this disclosure, the term “based on” means “based at least in part on.” The singular forms “a,” “an,” and “the” include plural referents unless the context dictates otherwise.

Embodiments of this disclosure relate generally to methods and systems for determining systemic vascular resistance (SVR) using a photoplethysmography (PPG) device. According to implementations of the disclosed subject matter, medical condition treatment and patient outcomes can be improved by monitoring SVR iteratively and/or continuously, and/or treating a medical condition based on the same. A medical condition may be treated based on monitoring and/or adjusting the administration of drugs and/or the addition or removal of body fluids, such as augmentation through infusion, or removal by drugs or dialysis, or the like, based on SVR. Patients with cardiac pacemakers form a subset of the population who suffer from a form of cardiovascular illness. For example, approximately 70% of patients with a pacemaker may have hypertension. Accordingly, the availability of real time continuous SVR measurements may be used for a diagnosis and/or therapy for such patients. The availability of SVR measurements, particularly in a continuous reliable data stream, as disclosed herein, can enhance prevention and therapy of medical conditions such as the hypertension or heart failure.

The term “photoplethysmography device” (“PPG” device) may be a light based device and/or may use different techniques to measure the changes in blood flow or volume (e.g., light, pulse transit time measurements, ultrasound, magnetic resonance imaging, indicator dilution methods, intravenous injection of contrast for X-ray imaging, thermography, estimates of capillary filling, etc.). The PPG device may be a wearable device, e.g., a watch, a band, a strap, etc., or a non-wearable device. A PPG device may operate by measuring the “pulse transit time,” which is converted to a respective blood pressure. The pulse transit time measures the time it takes for blood to move from a first part of an artery to a second part of an artery. A PPG device may use a non-invasive optical method for measuring blood volume changes per pulse. A PPG waveform output by a PPG device may represent the mechanical activity of the heart. Blood pressure may be determined by analysis of the PPG waveform. A PPG measurement may be subject to imprecision from a number of factors, including but not limited to, calibration issues, effects on blood pressure based on arm positions (e.g., from the variable contribution of gravity), and/or local vasospasm effects on blood flow such as cold temperature, etc. The blood pressure measurement itself, although improved by the use of light-emitting diodes, suffers inherent drift with prolonged use. For these reasons and more, a means of calibrating a PPG device, and other indirect blood pressure measurement apparatuses not including an oscillometer is disclosed herein.

As further disclosed herein, a PPG device or an associated processor or device may utilize a machine learning model to correct for the effect of gravitational forces on the PPG device's measured blood pressure. As discussed in more detail below, in various embodiments, systems and methods are described for using machine learning to correct for the effect of gravitational forces on the PPG device's measured blood pressure. By training a machine learning model, e.g., via supervised, semi-supervised, or unsupervised learning, to learn associations between PPG device location and/or height and blood pressure measurements, the trained machine learning model may be used to correct for the effect of gravitational forces on blood pressure. As discussed herein, there may be numerous benefits to calibrating a PPG device for gravitational changes, such as increased accuracy in medical diagnoses, more effective medical treatments, etc.

According to some embodiments, a continuous method of blood pressure measurement may be implemented using a photoplethysmography (PPG) device, e.g., a calibrated PPG device. A PPG device may receive or determine a systolic blood pressure (SBP), diastolic blood pressure (DBP), and a pulse transit time (PTT). Indirect measurement of cardiac output may be determined using PTT, and indirect measurement of mean arterial pressure (MAP) may be determined using the SBP and DBP. A cardiac output (CO) may be determined by multiplying an obtained heart rate by a stroke volume estimated using the PTT output by the calibrated PPG device. By combining the output of both measurement techniques—PPG device-derived PTT to estimate cardiac output/stroke volume and blood pressure to estimate left or right ventricular stroke volume—a continuous measurement of SVR can be made. A first value may be determined by subtracting one of (i) a right atrial pressure (RAP) or (ii) a central venous pressure (CVP) from the MAP. A second value may be determined by dividing the first value by the cardiac output CO. An estimated SVR may be determined by multiplying the second value by a factor (e.g., approximately 80).

As discussed herein, blood pressure (BP) may be one or both of systolic or diastolic blood pressure. Other acronyms used herein include, heart rate (HR), cardiac output (CO), and right or left ventricular stroke volume (SV). A blood pressure may indicate how much pressure a user's blood exerts against the user's artery walls when the user's heart beats (e.g., a systolic blood pressure). A blood pressure may indicate how much pressure a user's blood exerts against the user's artery walls when the user's heart is resting between beats (e.g., diastolic blood pressure).

The term “algorithm,” as used herein, refers to a sequence of defined computer-implementable instructions, typically to solve a class of problems or to perform a computation. Terms such as “noise,” or the like, as used herein, generally encompass extraneous, irrelevant, or relatively less meaningful data, or any data that is other than a signal intended to be observed. In the case of waveforms, “noise” may include, for example, unwanted signals that are merged with the waveform signal. Terms such as “signal” or the like, a used herein, generally encompass any function that may convey information about a phenomenon. “Signals” may refer to any time varying voltage, current, or electromagnetic wave that carries information or an observable change in a quality, such as quantity, or may refer to the information itself.

As used herein, a “machine learning model” generally encompasses instructions, systems, data, and/or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine learning model is generally trained using training data, e.g., experiential data and/or samples of input data, which are fed into the model in order to establish, tune, or modify one or more aspects of the model, e.g., the weights, layers, biases, criteria for forming classifications or clusters, or the like. Aspects of a machine learning model may operate on an input linearly, in parallel, via a network (e.g., a neural network), or via any suitable configuration.

The execution of the machine learning model may include deployment of one or more machine learning techniques, such as linear regression, logistical regression, random forest, gradient boosted machine (GBM), deep learning, and/or a deep neural network. Supervised and/or semi-supervised training may be employed. For example, supervised learning may include providing training data and labels corresponding to the training data, e.g., as ground truth. Semi-supervised approaches may include heuristic, generative, low-density, Laplacian or other like models. K-means clustering or K-Nearest Neighbors may also be used, which may be supervised or semi-supervised. Combinations of K-Nearest Neighbors and a semi-supervised cluster technique may also be used. Any suitable type of training may be used, e.g., stochastic, gradient boosted, random seeded, recursive, epoch or batch-based, etc.

Terms like “provider,” “medical provider,” or the like generally encompass an entity, person, or organization that may seek information, resolution of an issue, or engage in any other type of interaction with a user, e.g., to provide medical care, medical intervention or advice, or the like. Terms like “user,” “patient,” or the like generally encompass any person (e.g., an individual, a medical provider, etc.) or entity who is using a device, calibrating a device, obtaining information, seeking resolution of an issue, or the like.

As disclosed herein, a gold standard device may be a device used to conduct a gold standard test for calibration. A gold standard test may be a diagnostic test or benchmark that is the best available under reasonable conditions. A gold standard device may be one that has been tested and has a reputation in the field as a reliable method. For example, a gold standard device may include, but is not limited to, a device that uses a column of mercury (e.g., in a cylinder, such as glass) to determine a blood pressure. The gold standard device may detect a force of blood necessary to raise mercury column a known amount at sea level in the Earth's gravitational field.

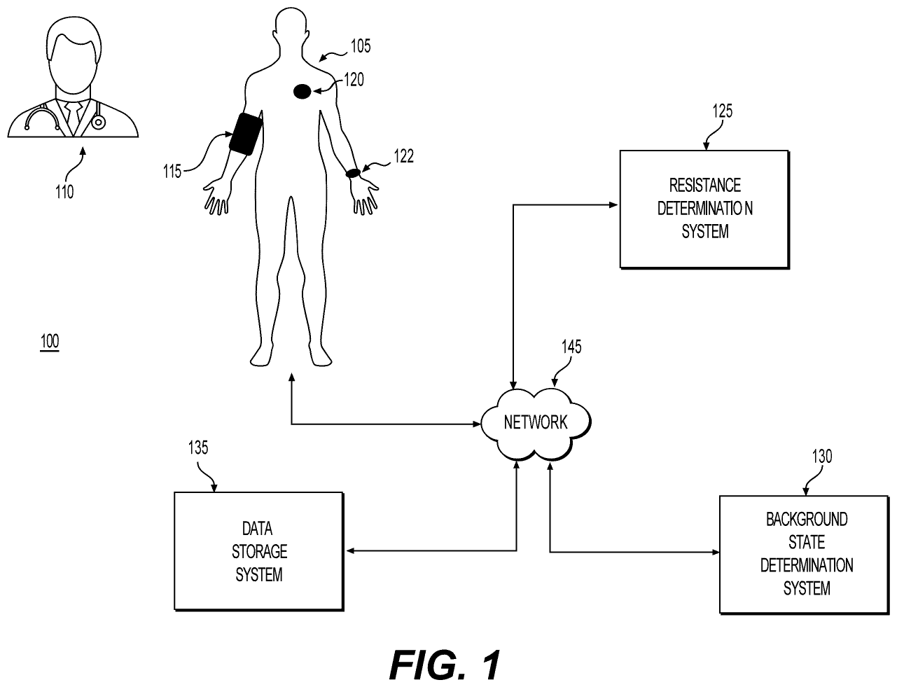

According to implementations of the disclosed subject matter, as shown in the environment 100 of FIG. 1, a user 105 and/or a medical provider 110 may operate a gold standard blood pressure device 115, a pacemaker 120 (e.g., a heart rate measuring device), and/or a photoplethysmography (PPG) device 122. User 105 may wear any of gold standard blood pressure device 115, pacemaker 120, and/or PPG device 122 simultaneously, or any combination thereof at a time. The results from gold standard blood pressure device 115, pacemaker 120, and/or PPG device 122 may be transmitted via a network 145 between one or more of resistance determination system (hereinafter “resistance system”) 125, background state determination system (hereinafter “background state system”) 130, data storage system 135, etc.

Gold standard blood pressure device 115, pacemaker 120, and/or PPG device 122 may operate continuously, at intervals, or at the determination of user 105, medical provider 110, and/or a user. Gold standard blood pressure device 115, pacemaker 120, and/or PPG device 122 may include one or more sensors either internal or external to the respective device. For example, pacemaker 120 may include at least one electrode. Pacemaker 120 may be a single chamber pacemaker with a right ventricular lead, a biventricular pacemaker, a dual chamber pacemaker, a biventricular cardio-defibrillator, or the like. The blood pressure devices discussed herein, e.g., gold standard blood pressure device 115, PPG device 122, etc., may be a blood pressure monitor that may be implemented as a standalone blood pressure monitoring device or may be incorporated in a fitness tracker, wearable device, digital watch, digital band, patch, arm cuff, a PPG device, or the like. PPG device 122 may be further configured to measure a PTT, e.g., via a pulse oximeter.

According to implementations of the disclosed subject matter, blood pressure may be measured discretely such as by a single cuff inflation method of gold standard blood pressure device 115. A bioimpedance may be measured from one or more electrodes or leads of pacemaker 120. Alternatively, or in addition, bioimpedances may be measured from one or more leads of pacemaker 120 and augmented by additional skin electrodes (not depicted) connected by hardwires or wireless methods. All measurements disclosed herein may be augmented, trended, and/or individualized using artificial intelligence, such as machine learning. According to an implementation, skin sensing techniques may be augmented beyond the capabilities of standard electrocardiogram (EKG) electrodes using solid-state sensing elements, either silicon-based, metal film, non-aqueous polymeric plastic with immobilized ions, or field effect transistor methods.

According to embodiments disclosed herein, resistance system 125 may be configured to determine an SVR. Resistance system 125 may be configured to obtain at least one of a SBP, a DBP, two or more bioimpedances (e.g., a first bioimpedance, a second bioimpedance, etc.), a heart rate, etc. from aspects of environment 100. Based on the data obtained, resistance system 125 may be configured to determine at least one of a SV, a MAP, a CO, and more. Resistance system 125 may be configured to transmit data (e.g., the obtained data, the determined data, etc.) to any suitable aspect of environment 100. According to implementations of the disclosed subject matter, SVR may be determined by resistance system 125 using one or more blood pressure and PTT to estimate right or left ventricular SV. Artificial intelligence such as machine learning may be used to output the SVR or to output one or more modified or corrected calibration factors, blood pressures, SV estimates, bioimpedances, or the like.

The machine learning model may analyze data received from user 105, provider 110, gold standard blood pressure device 115, pacemaker 120, PPG device 122, data storage system 135, and/or any other person, entity, or device. For example, data from gold standard blood pressure device 115 (e.g., blood pressure data), pacemaker 120 (e.g., pacemaker data), and/or PPG device 122 (e.g., PTT data) may be input into the machine learning model. The trained machine learning model may correlate a SBP, a DBP, two or more bioimpedances (e.g., a first bioimpedance, a second bioimpedance, etc.), a PTT, a heart rate, a SV, a MAP, a CO, and other data to determine SVR. The machine learning model may output a SVR. In some examples, the trained machine learning model may be configured to filter one or more of an SBP, a DBP, a MAP, a RAP, a CVP, a first value, a second value, or an SVR for one or more of noise reduction, stabilization, or amplification (e.g., signal amplification).

In various embodiments, a processor or storage component (e.g., data storage system 135), gold standard blood pressure device 115, pacemaker 120, and/or PPG device 122 may generate, store, train, or use the machine learning model and/or may include instructions associated with the machine learning model, e.g., instructions for generating the machine learning model, training the machine learning model, using the machine learning model, etc. For example, blood pressure measured using gold standard blood pressure device 115 may be transmitted, via a Bluetooth protocol, to a processor associated with PPG device 122. The processor may receive the measured blood pressure to output the SVR or one or more modified or corrected blood pressures, calibration factors, SV estimates, bioimpedances, or the like. The processor or one or more other processors may apply the modified or corrected values to more accurately determine the SVR.

In some embodiments, a system or device other than gold standard blood pressure device 115, pacemaker 120, or PPG device 122 may be used to generate and/or train the machine learning model. For example, such a system may include instructions for generating the machine learning model, the training data and ground truth, and/or instructions for training the machine learning model. Training data may include one or more of historical blood pressures, historical bioimpedances, historical PTTs, historical SBPs, historical DBPs, historical MAPs, historical RAPs, historical CVPs, historical first values, historical second values, historical SVRs, and more from a plurality of users. A resulting trained machine learning model may then be provided to PPG device 122 or a component associated with PPG device 122 such that the trained machine learning model can output an SVR and/or one or more modified or corrected calibration factors, blood pressures, SV estimates, bioimpedances, etc.

Generally, a machine learning model includes a set of variables, e.g., layers, nodes, neurons, filters, weights, biases, etc., that are tuned, e.g., weighted or biased, to different values via the application of training data. In supervised learning, e.g., where a ground truth is known for the training data provided, training may proceed by feeding a sample of training data into a model with variables set at initialized values, e.g., at random, based on Gaussian noise, a pre-trained model, or the like. The output may be compared with the ground truth to determine an error, which may then be back-propagated through the model to adjust the values of the variable.

Training may be conducted in any suitable manner, e.g., in batches, and may include any suitable training methodology, e.g., stochastic or non-stochastic gradient descent, gradient boosting, random forest, adaptive moment estimation (“ADAM”), etc. Training may be conducted with or without sample and/or class weighting. In some embodiments, a portion of the training data may be withheld during training and/or used to validate the trained machine-learning model, e.g., compare the output of the trained model with the ground truth for that portion of the training data. The training of the machine learning model may be configured to cause the machine learning model to learn associations between (i) gold standard data and/or PPG device data and (ii) gravitational effects based on device positioning, such that the trained machine learning model is configured to determine an output (e.g., corrected PPG device data) in response to the input data based on the learned associations. For example, the machine learning model may receive PPG device data points (e.g., blood pressure) associated with a particular arm positioning, which the machine learning model may be trained to correct based on the calibration factor applied to the arm positioning.

In various embodiments, the variables of a machine learning model may be interrelated in any suitable arrangement in order to generate the output. For example, in some embodiments, the machine learning model may include architecture that is configured to identify, isolate, and/or extract features, geometry, and or structure in input data. For example, the machine learning model may include one or more convolutional neural networks (“CNN”) configured to identify features in the signal-processed data, and may include further architecture, e.g., a connected layer, neural network, etc., configured to determine a relationship between the identified features in order to determine a location in the signal-processed data.

The sensitivity and specificity of the SVR determination technique disclosed herein may be further augmented by using a sensor device to detect background states (e.g., noise, motion, interference signals, etc.), e.g., by background state system 130. For example, a sensor device (e.g., a peripheral sensor device) such as a wrist bracelet, ankle bracelet, smartwatch, or the like may be used to determine background states. Such sensor device based measurements may expand the total volume of available measurements. Traditional devices for measurement of bioimpedance (e.g., such as the Optivol® system marketed by Medtronic, Inc.) suffer from a limited special vector volume that is confined to the area between the pacemaker lead tips and the pacemaker metal can. By adding additional spatial volume to the measurements discussed herein, such as by including a sensing device (e.g., a wireless bioimpedance-sensing bracelet) to the sensor network, artifact may be reduced and measurement sensitivity and specificity may proportionately increase.

Such traditional systems (e.g., the Optivol® system) also suffer from false positive total thoracic compartment fluid estimations (e.g., for diagnosis of heart failure) when the source of the fluid is confined to the lungs due to an inflammatory state and/or infection rather than heart failure. Such false positives are generally a drawback of traditional pacemaker lead-derived bioimpedance measurements, especially systems where the spatial vectors are limited to the area over or around the left lung tissue directly adjacent (e.g., as in the Optivol® system).

The sensor device may have wireless communication capabilities to communicate with a processor (e.g., a wearable device processor or related processor) to provide both a more stable background state not limited to the bioimpedance volume confined between pacemaker lead tips and a pacemaker can. Such a background state(s) determined using a sensor device may be used in addition to or instead of, for example, using a single sensing element on a pacemaker. According to this implementation, the background state may be used to isolate the contribution of thoracic fluid content as a separate variable, and to remove it as an artifact from a SVR determination. Accordingly, sensed data from a sensor device may be used to remove background state artifacts from one or more measurements disclosed herein, when determining an SVR. A reduction in the background noise or any artifact (e.g., motion artifact) may be implemented using systems and/or components intrinsic to a blood pressure device without using a sensor device. However, it will be understood that adding a sensor device (e.g., an additional vector or area of measurement) to a given ground may provide enhanced artifact correction.

According to embodiments disclosed herein, background state system 130 may be configured to filter (e.g., reduced, modified, and/or removed) one or more forms of noise (e.g., background states) found in one or more signals, e.g., bioimpedance, blood pressure, etc. The one or more signals may be generated using gold standard blood pressure device 115, pacemaker 120, and/or PPG device 122. A noise reduction algorithm may be used by background state system 130 to filter noise. The type of noise reduction algorithm may depend on the type of noise in the data, the type of data, or the like. The noise reduction algorithm may be automatically selected and/or applied, or may be selected and/or applied based on user input. A type of noise may include, but is not limited to, high frequency noise, movement noise, and/or any other form of noise. A plurality of noise reduction algorithms may be used to filter noise for a given signal. According to embodiments disclosed herein an amplification may be applied to amplify a signal generated at gold standard blood pressure device 115, pacemaker 120, and/or PPG device 122. Such signal amplification may be performed prior to, in conjunction with, or post filtering the signal for noise.

In some embodiments, the network 145 may connect one or more components of the environment 100 via a wired connection, e.g., a USB connection between gold standard blood pressure device 115, pacemaker 120, and/or the PPG device 122. In some embodiments, the network 145 may connect one or more aspects of the environment 100 via an electronic network connection, for example a Bluetooth connection, a wide area network (“WAN”), a local area network (“LAN”), personal area network (“PAN”), or the like. In some embodiments, the electronic network connection includes the Internet, and information and data provided between various systems occurs online. “Online” may mean connecting to or accessing source data or information from a location remote from other devices or networks coupled to the Internet. Alternatively, “online” may refer to connecting or accessing an electronic network (wired or wireless) via a mobile communications network or device. The Internet is a worldwide system of computer networks—a network of networks in which a party at one computer or other device connected to the network can obtain information from any other computer and communicate with parties of other computers or devices. The most widely used part of the Internet is the World Wide Web (often-abbreviated “WWW” or called “the Web”). A “website page,” a “portal,” or the like generally encompasses a location, data store, or the like that is, for example, hosted and/or operated by a computer system so as to be accessible online, and that may include data configured to cause a program such as a web browser to perform operations such as send, receive, or process data, generate a visual display and/or an interactive interface, or the like.

According to embodiments, environment 100 may be a closed loop such that no external network connection may be necessary to implement the techniques disclosed herein. The closed loop maybe used to provide a real-time automatic method that is self-contained and not dependent upon linkage to a remote server containing additional software, often referred to as “edge computing.” The method is also suitable for transmission to the cloud to allow for an interface with conventional electronic health records and other data analysis and reporting processes.

In such a closed loop system, as discussed herein, a blood pressure, a bioimpedance (e.g., a first bioimpedance, a second bioimpedance, etc.), and/or a PTT may be transmitted over a Bluetooth connection. One or more of pacemaker 120 and/or PPG device 122 may be associated with a processor that may apply a received and/or determined blood pressure, bioimpedance, heart rate, cardiac output, stroke volume, etc. to determine a systemic vascular resistance (e.g., using a machine learning model). Accordingly, the connections within the environment 100 can be wireless, wired, or be any other suitable connection, or any combination thereof.

In some embodiments, the data storage system 135 may store the data from and/or provide data to various aspects of the environment 100. Data storage system 135 may include a server system, an electronic medical data system, computer-readable memory such as a hard drive, flash drive, disk, etc. In some embodiments, data storage system 135 includes and/or interacts with an application programming interface for exchanging data to other systems, e.g., one or more of the other components of the environment 100. Data storage system 135 may include and/or act as a repository or source for data from gold standard blood pressure device 115, pacemaker 120, PPG device 122, medical history and/or diagnoses for user 105, machine learning data, and/or other forms of data. Data storage system 135 may be external to or may be a part of gold standard blood pressure device 115, pacemaker 120, and/or PPG device 122.

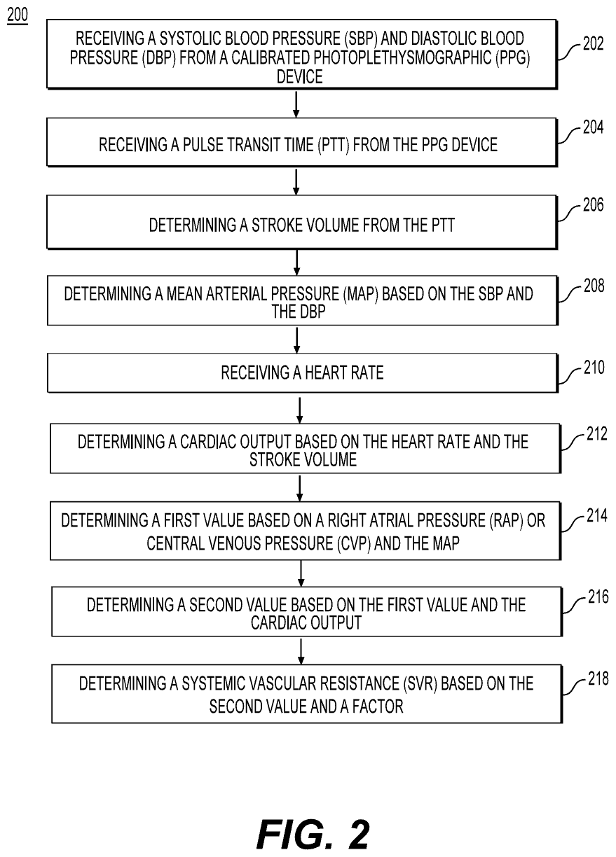

FIG. 2 shows a flow chart 200 for determining an SVR using PTT. According to an implementation, a calibrated continuous accurate method of blood pressure measurement may be implemented using PPG. According to an implementation, a PPG device (e.g., a blood pressure measurement device), such as PPG device 122 described in detail above, may be calibrated to output blood pressure readings that account for the position of the PPG device relative to a reference point (e.g., a user's heart). The PPG device calibration may be implemented using a gold standard device. To calibrate the PPG device, gold standard device blood readings at multiple locations or positions relative to the reference point may be determined. A calibration factor may be determined based on the gold standard device blood pressure readings at the multiple locations or positions, for a given user. For example, the calibration factor may be determined by sensing a first blood pressure when the blood pressure device (e.g., gold standard blood pressure device 115, pacemaker 120, PPG device 122, etc.) is at a first height, sensing a second blood pressure when the blood pressure device is at a second height, sensing a third blood pressure when the blood pressure device is at a third height, etc. The calibration factor may be a relationship between the relative position of a device (e.g., the first height, the second height, the third height, etc.) and a change in blood pressure reading based on the relative position (e.g., the first blood pressure, the second blood pressure, the third blood pressure, etc.). The calibration factor may be applied by a PPG device such that a PPG device may be calibrated based on its position relative to a reference point.

According to an implementation, the calibration may be implemented by using a motion sensor with communication (e.g., wireless communication) capability affixed to a user proximate to the PPG device. The motion sensor may be a stand-alone component or may be an integral component of a smartwatch with communication (e.g., wireless communication) capability that senses the motion of the patient's wrist at one or more positions (e.g., one or more points of a 180 degree arc). The one or more positions may be, for example, extending from the wrist hanging down at the patient's side (e.g., minus ninety degrees), level with the patient's heart (e.g., zero degrees), and/or with the wrist raised in full extension above the patient's head (e.g., plus 90 degrees). PPG calibration is further disclosed in International Application No. PCT/US2022/072989, filed Jun. 16, 2022, which is incorporated herein by reference in its entirety.

At step 202 of FIG. 2, an SBP and a DBP may be received from a PPG device, e.g., a calibrated PPG device. According to implementations of the disclosed subject matter, blood pressure may be measured and/or obtained by a calibrated PPG device. Accordingly, SVR may be determined by receiving calibrated blood pressures from a calibrated PPG device. The calibrated PPG device may be calibrated in accordance with the techniques disclosed herein. The calibrated PPG device may output an SBP and a DBP.

At step 204, a PTT may be received from the PPG device. According to implementations of the disclosed subject matter, an indirect method of estimating left or right ventricular stroke volume may be implemented by using a PTT. As discussed herein, Pulse Transit Time (PTT) refers to the time it takes a pulse wave to travel between two arterial sites. The speed at which this arterial pressure wave travels is directly proportional to blood pressure. As disclosed herein, PPG Pulse Transit Time to estimate left or right ventricular stroke volume may be used to determine a continuous measurement of SVR. PTT may be the time taken for an arterial pulse pressure wave to travel from the aortic valve to a peripheral site. PTT may be measured from an R wave on the electrocardiogram to the pulse wave arrival at the finger. For example, an electrocardiogram (EKG) electrode may output the R wave of an EKG so that the time between that R Wave and the onset of PPG wave's appearance may be measured. Such a measurement may be determined using a pacemaker that is a source of EKG signals.

According to an example implementation, an EKG sensor (e.g., electrode) may be integrated with or connected to a wearable device (e.g., a smart watch) or may be provided as a separate EKG lead and/or a cable. According to another example implementation, a wearable sensor or a wearable device with a sensor may include an EKG electrode configured to send EKG signals to a component (e.g., a processor, base unit, etc.) any other applicable signals (e.g., sensed data). Alternatively, or in addition, the wearable device and/or sensor may include a component (e.g., a processor) to determine the PTT in the based on an EKG signal and the wearable device and/or sensor may send the determined PTT to an applicable component (e.g., a processor, base unit, etc.).

Still referring to FIG. 2 at step 206, the PTT may be used to determine a stroke volume. The PTT, output by a calibrated PPG, may be used to estimate a stroke volume. At step 208, a MAP may be determined based on the SBP and the DBP (e.g., as shown in Equation 1). For example, MAP may be determined by the following Equation 1 using SBP and DBP: MAP=SBP+2 (DBP).

At step 210, a heart rate may be received from a heart rate measuring device. For example, the heart rate may be determined using a pacemaker or other heart rate sensing device. At step 212, a cardiac output may be determined based on the heart rate and the SV (e.g., by multiplying the heart rate by the SV). In some examples, cardiac output may be determined by using the estimate of stroke volume calculated using a PTT from a calibrated PPG multiplied by the heart rate.

At step 214, a first value may be determined based on one of (i) the RAP or (ii) the CVP, and the MAP (e.g., by subtracting the RAP or the CVP from the MAP). RAP may be directly measured (e.g., invasively) and may be in the range of approximately 8 mmHg to 12 mmHg as a mean number. RAP may be estimated clinically. During a clinical estimation, a patient may be placed supine on their side (e.g., left side) with the head at approximately 45 degrees. In this positon, the venous pulsations in the neck may be noted in cm from the level of the clavicle. Alternatively, the RAP may be estimated to be approximately 10 mmHg. Alternatively, or in addition, RAP may be measured indirectly using echocardiography. Normal SVR may be approximately 700 to 1,500 dynes/seconds/cm-5. CVP may be determined either directly by inserting a catheter into the body of the right atrium or may be indirectly estimated. The magnitude of CVP may be small compared to one or more other measurement parameters. Accordingly, an indirectly measured CVP may be estimated by assigning a mean value of 10 mmHg. The estimated CVP may be adjusted based on visual estimation of a Jugular Venous Pressure (e.g., if such Jugular Venous Pressure suggests a higher CVP value).

Still referring to FIG. 2 at step 216, a second value may be determined based on the first value and the cardiac output, e.g., by dividing the first value (determined at step 214) by the cardiac output (determined at step 212). At step 218, an SVR may be determined based on the second value and a factor (e.g., by multiplying the second value by approximately 80). The two derived measurements—blood pressure (systolic and diastolic) and cardiac output/stroke volume—may be combined to determine SVR (e.g., a continuous estimated SVR) by subtracting the right atrial pressure (RAP) or central venous pressure (CVP) from the mean arterial pressure (MAP) (see step 214), divided by the cardiac output (see step 216) and multiplied by a factor (e.g., approximately eighty).

The sensitivity and specificity of the SVR determination technique disclosed herein may be further augmented by using a sensor device to detect background states (e.g., noise, motion, interference signals, etc.). For example, a sensor device such as a wrist bracelet, ankle bracelet, smartwatch, or the like may be used to determine background states. The sensor device may have wireless communication capabilities to communicate with a processor (e.g., a PPG device processor or related processor) to provide both a more stable background state. Such a background state determined using a sensor device may be used in addition to or instead of, for example, using a single sensing element on a PPG device (e.g., a PPG smartwatch) platform. According to this implementation, the background state may be used to isolate the contribution of thoracic fluid content as a separate variable, and to remove it as an artifact from a SVR determination. Accordingly, a sensor device may be used to remove background state artifacts (e.g., noise, motion, interference signals, etc.) from one or more measurements disclosed herein, when determining an SVR. A reduction in the background noise or any artifact (e.g., motion artifact) may be implemented using systems and/or components intrinsic to a PPG device without using a sensor device. However, it will be understood that adding a sensor device (e.g., an additional vector or area of measurement) to a given ground may provide enhanced artifact correction.

As discussed herein, blood pressure (BP) may be one or both of systolic or diastolic blood pressure. Other acronyms used herein include, heart rate (HR), cardiac output (CO), and right or left ventricular stroke volume (SV). As further discussed herein, PPG is an optically obtained plethysmogram that may be used to detect blood volume changes in the microvascular bed of tissue. A PPG value may be obtained by using a pulse oximeter (e.g., a pulse oximeter of PPG device 122) that illuminates a user's skin and measures changes in light absorption. A pulse oximeter may monitor the perfusion of blood to the dermis and subcutaneous tissue of the skin.

A change in volume caused by a pressure pulse may be detected by illuminating the skin with the light from a light-emitting diode (LED) and measuring the amount of light either transmitted or reflected to a photodiode. Each cardiac cycle may appear as a peak. As blood flow to the skin can be modulated by multiple other physiological systems, a PPG can also be used to monitor breathing, hypovolemia, and other circulatory conditions. Additionally, the shape of the PPG waveform differs from subject to subject, and varies with the location and manner in which the pulse oximeter is attached.

Traditional continuous SVR measurements may be performed using an invasive approach, such as an arterial cannula. Traditional noninvasive measurements are serially generated several minutes apart (not continuously). During such noninvasive measurements, the compressive effects of a traditional blood pressure cuff may become progressively painful and influence the results by inducing peripheral artery spasm. Traditional system cannot calculate continuous SVR without a calibrated means to determine continuous blood pressure.

Traditional methods of PPG continuous blood pressure measurement do not include medical grade calibration against a gold standard blood pressure cuff (e.g., an FDA cleared cuff) that has been calibrated against a gold standard (e.g., a true mercury manometer). Moreover, resting PPG measurements using a smart watch with a subject's arm fixed to his/her side do not compensate for measurement deviations caused by the effect of gravity when the arm moves up or down with respect to the heart. The calibration techniques disclosed herein includes a continuous correction based on device (e.g., attached to an arm) position. The PPG calibration disclosed herein is further augmented using machine learning to individualize the calibration for each subject.

Implementations disclosed herein use estimated stroke volumes in conjunction with a calibrated PPG blood pressure measurement and heart rate (e.g., from a pacemaker or other heart rate detection device) to calculate SVR. The combination of the stroke volume and calibrated PPG blood pressure are used to implement the continuous SVR measurement techniques disclosed herein.

According to implementations disclosed herein, left or right ventricular SVR can be indirectly estimated using a calibrated PPG device based on a PTT calculation. The PPT calculation may be proportional to stroke volume when calibrated for sensor position relative to a reference point (e.g., the level of the heart), and with a filtered PTT signal corrected for body motion, other motion artifact, band pass filtering as required, and 5G (fifth generation) correction. The techniques disclosed herein use a calibrated PPG blood pressure measurement and heart rate to calculate SVR.

According to implementations of the disclosed subject matter, continuous calibrated blood pressures may be obtained using a calibrated PPG device, as disclosed herein. The calibrated blood pressures may be used to determine real-time continuous SVR in patients by estimating SV using PTT derived from the calibrated (e.g., position corrected) PPG device for diagnosing and treating conditions associated with abnormal SVR.

According to implementations of the disclosed subject matter, SVR may be estimated using a calibrated PPG device. The calibrated PPG device may be motion corrected. The calibrated PPG device may detect blood pressure (e.g., blood pressure values, one or more signals that are used to determine blood pressures, etc.). One or more subroutines may be implemented to correct or modify a PPG signal.

Such subroutines may include, but are not limited to band pass filters, motion artifact correction, 5G artifact correction, etc., or a combination thereof. A subroutine may measure PTT and estimate ventricular SV. A subroutine may calculate SVR based on the calibrated PPG blood pressure, corrected or modified PPG blood pressure signals, PTT and SV, as discussed herein.

According to implementations of the disclosed subject matter, data analysis, trends, calibration factors, or one or more other items disclosed herein may be output by and/or modified by a machine learning model. The machine learning model may be trained to modify or correct such items based on individual user data, cohort user data, historical data, changes in user states, trends, or the like.

According to implementations of the disclosed subject matter, wireless transmission techniques for transmitting and/or receiving items disclosed herein may be implemented. Sensed data, estimated data, blood pressure values, SV, SVR, etc. may be wirelessly transmitted and/or received via a network, as further described herein. Such data may be stored locally or at a remote storage or memory such as in a cloud component. The cloud component or other storage or memory may be implemented using security protocols configured to mitigate loss of data, to mask data, to encrypt data, or the like.

According to implementations of the disclosed subject matter, blood pressure may be measured discretely such as by a single cuff inflation method, or continuously such as by PPG techniques. Left or right ventricular SV may be measured using PPG-derived PTT to estimate left or right ventricular SV. All measurements may be augmented, trended, and/or individualized using artificial intelligence, such as machine learning. According to an implementation, skin sensing techniques may be augmented beyond the capabilities of standard EKG electrodes using are solid-state sensing elements, either silicon-based, metal film, or non-aqueous polymeric plastic with immobilized ions.

The techniques disclosed herein may be implemented via a bench top integration and testing of the components disclosed herein. A prototype may be tested in an anesthetized animal model, and then validated in humans in a cardiac catheterization laboratory in parallel with regular diagnostic catheterization carried out in consenting patient subjects.

The PPG device discussed herein may be a blood pressure monitor that may be implemented as a standalone blood pressure monitoring device or may be incorporated in a fitness tracker, wearable device, digital watch, digital band, patch, or the like.

According to implementations of the disclosed subject matter, SVR may be determined by using a calibrated PPG to measure blood pressure and the calibrated PPG to estimate right or left ventricular SV using PTT calculated from the calibrated PPG signal. Artificial intelligence such as machine learning may be used to output the SVR or to output one or more modified or corrected blood pressures, SV estimates, PTT measurements, or the like. Component signals from the calibrated PPG may be pre-processed and/or post processed to include motion artifact reduction, band pass filtering (e.g., for noise reduction, stabilization, amplification, etc.), position correction, 5G correction, and/or the like. Component signals from the calibrated PPG may be analyzed as discrete time/event data, and/or continuous domain data. One or more data disclosed herein (e.g., calculations, analysis, measurements, signals, estimates, etc.) may be maintained off site, such as at a cloud component with secure wireless communication and/or storage.

Techniques disclosed herein may be implemented in a closed-loop therapeutic system to diagnose and/or treat medical conditions including, but not limited to, hypertension and heart failure. The closed loop system may be implemented in real-time and/or autonomously utilizing all or part of the data discussed herein, including but not limited to SVR, and may include real-time patient interaction with one or more control algorithm(s).

Data that is output, calculated, sensed, determined, or otherwise discussed herein, such as SVR, or any other hemodynamic variables including but not limited to HR, CO, SV, cardiac rhythm, PTT, etc. may be provided to and/or used by any applicable diagnostic or therapeutic system, such as a remote or mobile patient treatment device, an entity, a system, a component, or the like or a combination thereof.

Techniques disclosed herein may be used to regulate a cardiac pacemaker, cardio-defibrillator, combination device, or other bodily implant for the diagnosis or treatment of disease. Said regulation may be implemented in a real-time automatic closed loop system. Alternatively, or in addition, such regulation may be implemented using a component in a decision chain that is not a closed loop in design, with manual data entry or control points operated by an operator such as a user, a health care provider, or the like (e.g., on site or remotely).

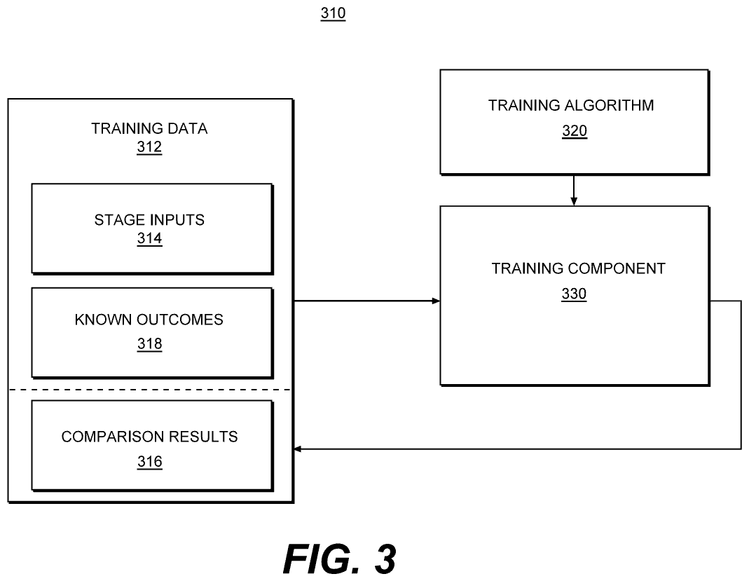

One or more implementations disclosed herein may be applied by using a machine learning model. For example, a machine learning model may be used to determine a state machine and/or a next state. As shown in flow diagram 310 of FIG. 3, training data 312 may include one or more of stage inputs 314 and known outcomes 318 related to a machine learning model to be trained. The stage inputs 314 may be from any applicable source including an input or system discussed herein (e.g., an output from a step of FIG. 2). The known outcomes 318 may be included for machine learning models generated based on supervised or semi-supervised training. An unsupervised machine learning model might not be trained using known outcomes 318. Known outcomes 318 may include known or desired outputs for future inputs similar to or in the same category as stage inputs 314 that do not have corresponding known outputs.

The training data 312 and a training algorithm 320 may be provided to a training component 330 that may apply the training data 312 to the training algorithm 320 to generate a machine learning model. According to an implementation, the training component 330 may be provided comparison results 316 that compare a previous output of the corresponding machine learning model to apply the previous result to re-train the machine learning model. The comparison results 316 may be used by the training component 330 to update the corresponding machine learning model. The training algorithm 320 may utilize machine learning networks and/or models including, but not limited to a deep learning network such as Deep Neural Networks (DNN), Convolutional Neural Networks (CNN), Fully Convolutional Networks (FCN) and Recurrent Neural Networks (RCN), probabilistic models such as Bayesian Networks and Graphical Models, and/or discriminative models such as Decision Forests and maximum margin methods, or the like.

In general, any process or operation discussed in this disclosure that is understood to be computer-implementable, such as the flows and/or process discussed herein (e.g., in FIGS. 1-3), etc., may be performed by one or more processors of a computer system, such any systems or devices used to implement the techniques disclosed herein. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer system. A processor may be a central processing unit (CPU), a graphics processing unit (GPU), or any suitable types of processing unit.

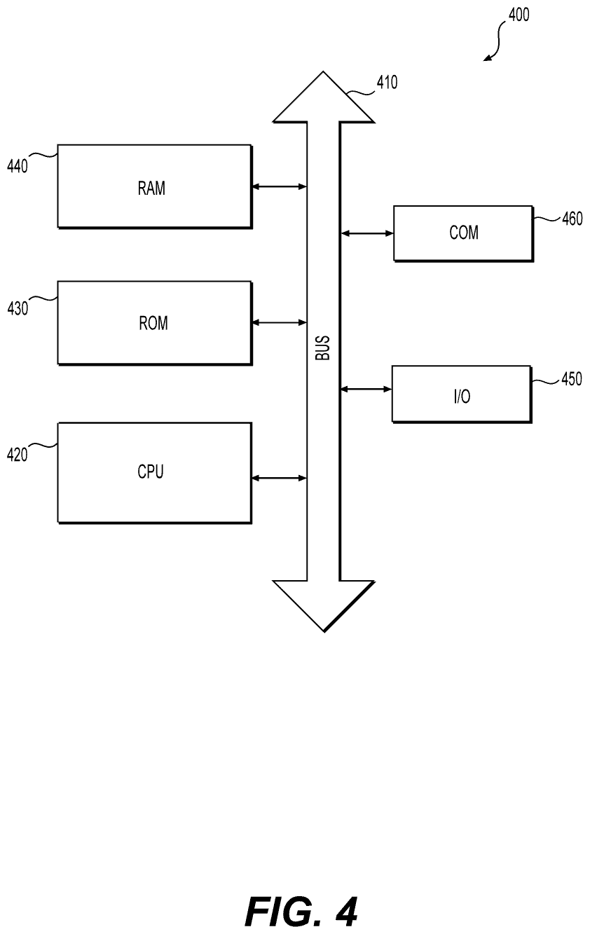

FIG. 4 depicts an example system 400 that may execute techniques presented herein. FIG. 4 is a simplified functional block diagram of a computer that may be configured to execute techniques described herein, according to exemplary embodiments of the present disclosure. Specifically, the computer (or “platform” as it may not be a single physical computer infrastructure) may include a data communication interface 460 for packet data communication. The platform may also include a central processing unit (“CPU”) 420, in the form of one or more processors, for executing program instructions. The platform may include an internal communication bus 410, and the platform may also include a program storage and/or a data storage for various data files to be processed and/or communicated by the platform such as ROM 430 and RAM 440, although the system 400 may receive programming and data via network communications. The system 400 also may include input and output ports 450 to connect with input and output devices such as keyboards, mice, touchscreens, monitors, displays, etc. Of course, the various system functions may be implemented in a distributed fashion on a number of similar platforms, to distribute the processing load. Alternatively, the systems may be implemented by appropriate programming of one computer hardware platform.

The general discussion of this disclosure provides a brief, general description of a suitable computing environment in which the present disclosure may be implemented. In one embodiment, any of the disclosed systems, methods, and/or graphical user interfaces may be executed by or implemented by a computing system consistent with or similar to that depicted and/or explained in this disclosure. Although not required, aspects of the present disclosure are described in the context of computer-executable instructions, such as routines executed by a data processing device, e.g., a server computer, wireless device, and/or personal computer. Those skilled in the relevant art will appreciate that aspects of the present disclosure can be practiced with other communications, data processing, or computer system configurations, including: Internet appliances, hand-held devices (including personal digital assistants (“PDAs”)), wearable computers, all manner of cellular or mobile phones (including Voice over IP (“VoIP”) phones), dumb terminals, media players, gaming devices, virtual reality devices, multi-processor systems, microprocessor-based or programmable consumer electronics, set-top boxes, network PCs, mini-computers, mainframe computers, and the like. Indeed, the terms “computer,” “server,” and the like, are generally used interchangeably herein, and refer to any of the above devices and systems, as well as any data processor.

Aspects of the present disclosure may be embodied in a special purpose computer and/or data processor that is specifically programmed, configured, and/or constructed to perform one or more of the computer-executable instructions explained in detail herein. While aspects of the present disclosure, such as certain functions, are described as being performed exclusively on a single device, the present disclosure may also be practiced in distributed environments where functions or modules are shared among disparate processing devices, which are linked through a communications network, such as a Local Area Network (“LAN”), Wide Area Network (“WAN”), and/or the Internet. Similarly, techniques presented herein as involving multiple devices may be implemented in a single device. In a distributed computing environment, program modules may be located in both local and/or remote memory storage devices.

As discussed herein, a memory may include a device or system that is used to store information for immediate use in a computer or related computer hardware and digital electronic devices. Contents of memory can be transferred to storage (e.g., via virtual memory). Memory may be implemented as semiconductor memory, where data is stored within memory cells built from MOS transistors on an integrated circuit. Semiconductor memory may include volatile and/or non-volatile memory. Examples of non-volatile memory include flash memory and read-only memory (ROM), programmable read-only memory (PROM), erasable programmable read-only memory (EPROM), electrically erasable programmable read-only memory (EEPROM) memory, and the like. Examples of volatile memory include primary memory such as dynamic random-access memory (DRAM) and fast CPU cache memory such as static random-access memory (SRAM).

Aspects of the present disclosure may be stored and/or distributed on non-transitory computer-readable media, including magnetically or optically readable computer discs, hard-wired or preprogrammed chips (e.g., EEPROM semiconductor chips), nanotechnology memory, biological memory, or other data storage media. Alternatively, computer implemented instructions, data structures, screen displays, and other data under aspects of the present disclosure may be distributed over the Internet and/or over other networks (including wireless networks), on a propagated signal on a propagation medium (e.g., an electromagnetic wave(s), a sound wave, etc.) over a period of time, and/or they may be provided on any analog or digital network (packet switched, circuit switched, or other scheme).

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

The terminology used above may be interpreted in its broadest reasonable manner, even though it is being used in conjunction with a detailed description of certain specific examples of the present disclosure. Indeed, certain terms may even be emphasized above; however, any terminology intended to be interpreted in any restricted manner will be overtly and specifically defined as such in this Detailed Description section. Both the foregoing general description and the detailed description are exemplary and explanatory only and are not restrictive of the features, as claimed.

Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

1. A method for determining a systemic vascular resistance (SVR), the method comprising:
receiving a systolic blood pressure (SBP) and a diastolic blood pressure (DBP) from a calibrated photoplethysmography (PPG) device;
receiving a pulse transit time (PTT) from the calibrated PPG device;
determining a stroke volume based on the PTT;
determining a mean arterial pressure (MAP) based on the SBP and the DBP;
receiving a heart rate;
determining a cardiac output based on the heart rate and the stroke volume;
determining a first value based on a right atrial pressure (RAP) or central venous pressure (CVP) and the MAP;
determining a second value based on the first value and the cardiac output; and
determining the systemic vascular resistance (SVR) based on the second value and a factor.

2. The method of claim 1, wherein the calibrated PPG device is calibrated based on a calibration factor.

3. The method of claim 2, wherein the calibration factor is determined by:
sensing a first blood pressure by a first device when the first device is at a first height;
sensing a second blood pressure by the first device when the first device is at a second height, the second height being different than the first height; and
generating the calibration factor based on the first blood pressure, the first height, the second blood pressure, and the second height.

4. The method of claim 1, further comprising a machine learning model configured to generate a machine learning output to individualize at least one of the SBP, the DBP, the MAP, the RAP, the CVP, the first value, the second value, or the SVR.

5. The method of claim 4, wherein the machine learning model is trained using training data including one or more of historical blood pressures, historical bioimpedances, historical PTTs, historical SBPs, historical DBPs, historical MAPs, historical RAPs, historical CVPs, historical first values, historical second values, or historical SVRs for a plurality of users.

6. The method of claim 1, wherein the PTT is determined based on an electrocardiogram signal.

7. The method of claim 1, wherein the PTT is corrected for at least one of body motion, other motion artifact, or band pass filtering.

8. The method of claim 1, wherein one or more of the SBP, the DBP, the MAP, the RAP, the CVP, the first value, the second value, or the SVR are filtered for one or more of noise reduction, stabilization, or amplification.

9. The method of claim 1, wherein the heart rate is received from a pacemaker.

10. A system for determining a systemic vascular resistance (SVR) using photoplethysmography (PPG), the system comprising:
at least one memory storing instructions; and
at least one processor executing the instructions to perform a process, the at least one processor configured to:
receiving a systolic blood pressure (SBP) and a diastolic blood pressure (DBP) from a calibrated photoplethysmography (PPG) device;
receiving a pulse transit time (PTT) from the calibrated PPG device;
determining a stroke volume based on the PTT;
determining a mean arterial pressure (MAP) based on the SBP and the DBP;
receiving a heart rate;
determining a cardiac output based on the heart rate and the stroke volume;
determining a first value based on a right atrial pressure (RAP) or central venous pressure (CVP) and the MAP;
determining a second value based on the first value and the cardiac output; and
determining the SVR based on the second value and a factor.

11. The system of claim 10, wherein the at least one processor is configured to calibrate the calibrated PPG device based on a calibration factor.

12. The system of claim 11, wherein the at least one processor is configured to determine the calibration factor by:
sensing a first blood pressure by a first device when the first device is at a first height;
sensing a second blood pressure by the first device when the first device is at a second height, the second height being different than the first height; and
generating the calibration factor based on the first blood pressure, the first height, the second blood pressure, and the second height.

13. The system of claim 10, the system further comprising a machine learning model configured to generate a machine learning output to individualize at least one of the SBP, the DBP, the MAP, the RAP, the CVP, the first value, the second value, or the SVR.

14. The system of claim 13, wherein the at least one processor is configured to train the machine learning model using training data including one or more of historical blood pressures, historical bioimpedances, historical PTTs, historical SBPs, historical DBPs, historical MAPs, historical RAPs, historical CVPs, historical first values, historical second values, or historical SVRs for a plurality of users.

15. The system of claim 10, wherein the PTT is determined based on an electrocardiogram signal.

16. The system of claim 10, wherein the at least one processor is configured to correct the PTT for at least one of body motion, other motion artifact, or band pass filtering.

17. The system of claim 10, wherein the at least one processor configured to filter one or more of the SBP, the DBP, the MAP, the RAP, the CVP, the first value, the second value, or the SVR for one or more of noise reduction, stabilization, or amplification.

18. The system of claim 10, wherein the heart rate is received from a pacemaker.

19. A method for determining a systemic vascular resistance (SVR), the method comprising:
determining a mean arterial pressure (MAP) based on a systolic blood pressure (SBP) and a diastolic blood pressure (DBP) received from a calibrated photoplethysmography (PPG) device;
receiving a heart rate from a pacemaker and a pulse transit time (PTT) from the calibrated PPG device;
determining a stroke volume based on the PTT;
determining a cardiac output (CO) based on the heart rate and the stroke volume;
determining a first value based on a right atrial pressure (RAP) or central venous pressure (CVP) and the MAP;
determining a second value based on the first value and the CO; and
determining the SVR based on the second value and a factor.

20. The method of claim 19, further comprising:
generating a machine learning output by a machine learning model to individualize at least one of the PTT, the SBP, the DBP, the MAP, the RAP, the CVP, the first value, the second value, or the SVR.
